(19) United States
(12) Patent Application Publication
(10) Pub. No.: US 2016/0089294 A1
(43) Pub. Date: Mar. 31, 2016

(54) DUAL-CHAIR ASSEMBLY

(71) Applicant: Marc Jonas Guillaume, Hollywood, FL (US)

(72) Inventor: Marc Jonas Guillaume, Hollywood, FL (US)

(21) Appl. No.: 14/498,206

(22) Filed: Sep. 26, 2014

(51) Int. Cl. A61G 15/08; A47C 1/06; A61B 19/02; A61G 15/16; A61G 15/00; A61N 2/02

(52) U.S. Cl. CPC A61G 15/08; A61G 15/007; A61N 2/02; A61B 19/0256; A61G 15/005; A61G 15/16; A47C 1/06; A61B 2019/0257

(57) ABSTRACT

A dual-chair assembly is disclosed having a practitioner chair rotatably coupled to a base by an extension arm, the base fixedly attached to a ground surface, and disposed a separation distance from the practitioner chair. The dual-chair assembly also includes a subject chair coupled to and supported by the base. A practitioner control is communicatively coupled to practitioner chair actuators, the practitioner control including a user input interface operably configured for receiving a practitioner input, and, as a result of the practitioner input, causing the practitioner chair actuators to perform at least one of rotating the practitioner chair about the subject chair; adjusting an elevation of the practitioner chair; and rotating at least one of a roll angle, a pitch angle, and a yaw angle of the practitioner chair.

### FIELD OF THE INVENTION

The present invention relates generally to a dual-chair assembly, and more particularly relates to an ergonomic dual-chair assembly for use during treatment, the assembly including a practitioner chair coupled to a subject's chair.

### BACKGROUND OF THE INVENTION

Past inventions have demonstrated a variety of seating arrangements for medical, dental, grooming, spa, barber, and cosmetology services. The typical device is configured as a single chair. The practitioner's chair is not connected to the patient/subject's chair and is usually a standard office-type chair with a castor base. Such chairs are limited in their ability to assist practitioners with maintaining good posture and musculoskeletal health while performing treatment procedures on the subject. For example, practitioners are often required to lean over in a hunched or otherwise uncomfortable position, with wrists bent at an awkward angle, in order to reach certain areas of the subject during treatment. Treatment will often require the practitioner to maintain such positioning for a prolonged period of time, resulting in physical stress to the practitioner. The result of this deficiency in the prior art is a lack of functional coordination between subject and practitioner chairs, resulting in a static therapy environment that does not address the physical stress of the chair occupants. Additionally, current practitioner chairs do not include provisions for massage stimulation, which rejuvenates fatigued muscles resulting from prolonged static posture and enhances circulation.

In particular, studies have found a high prevalence of musculoskeletal problems in practitioners whose occupation requires prolonged static postures (PSP). Static posture refers to the way in which an individual holds his or her body or assumes certain positions over a prolonged time period, such as sitting, standing, gripping, and the like. Such physical exertions place increased loads or forces on the muscles and tendons, which contribute to fatigue. Moreover, holding extreme postures places very high static loads on the body, resulting in rapid fatigue. The cumulative effect of the time spent in certain positions can lead to prolonged static-posture damage to the musculoskeletal systems of the body.

For example, studies have found a high prevalence of musculoskeletal problems in dentists, with 64% reporting back aches and 58% reporting headaches during any given month. Similar health problems have been reported among dental hygienists. Studies suggest that the prevalence and location of pain and other symptoms such as headaches may be influenced by posture and work habits.

Webster Dictionary defines good posture as “the state of muscular and skeletal balance which protects the supporting structures of the body against injury or progressive deformity irrespective of the attitude in which these structures are working or resting.” Good posture results in uniform dispensing of gravitational force across the body so that no single part is under any undue pressure for a prolonged period. By maintaining a good posture, the entire body is in a state of equilibrium. It is especially beneficial for the spinal cord as it helps in proper travel of the nerve signals to the various body parts.

For dentists and hygienists who sit on wheeled stools when treating dental patients, or other practitioners, such as barbers and cosmetologists who stand and lean forward when servicing patrons, posture has always been a challenge. According to C.H.E.K. Institute, experts in the field of corrective kinesiology, “[f]or every inch that the head's center of gravity is shifted forward, the lower cervical spine is subsequently subjected to compressive forces equivalent to a one-time additional weight of the head.” Most heads weigh between 12 and 20 pounds, so if one's head were 3 inches forward, that would put between 36 and 60 pounds of extra weight on the lower cervical (neck) spine, which is extremely fatiguing to the muscles at the back of the neck. The pull on the back of the head is also known to cause chronic headaches. These muscles that must deal with this constant load become exhausted, and their blood flow proportionally diminishes as the muscle contraction increases. This lack of blood flow is often the primary source of muscle pain in the neck region. Over time, the pain may migrate lower as more muscles become involved. As certain muscles tighten under the strain, their opposites lengthen and weaken, extending the problem further into the body. Musculoskeletal pain, particularly back pain, has been found to be a major health problem for dental practitioners. For example, a study from Australia found that 82% of dentists reported at least one musculoskeletal symptom in the month that the study was conducted, and 64% reported back aches during the previous month. Similar health problems have also been reported during studies of dentists in the United States of America. As can be expected, such health problems result in increased sick leave and reduced productivity during working hours.

Additionally, hand, wrist, and arm pain are much more prevalent among dental professionals than the general public, due to the sustained grips and prolonged awkward postures that dentists and hygienists must employ on a daily basis. One study found that dentists are as much as four times more likely to experience hand, wrist, and/or arm pain than the general working public. Another study found that between 40 and 70 percent of dental professionals suffer from chronic hand and wrist pain, which affects their quality of life as well as their career longevity. Dental professions are generally at risk for carpal tunnel syndrome (CTS), an injury usually resulting from the swelling around the tendons and pinching of the median nerve, causing painful tingling, lack of muscle strength and control in the hand, and pain shooting from the hand up to the shoulder. CTS symptoms often are not indicative of a structural problem in the hand and wrist, rather the problem originates in the muscles/tendons that stabilize the arm during repetitive work.

In short, practitioners who have been practicing for any prolonged period of time have experienced the challenge of finding, and maintaining an ideal position and posture for treating patients, whether standing over a patient or subject, or leaning forward from a stool with castors. After many long procedures day in and day out, practitioners will frequently suffer from head, neck, wrist, shoulder, or back pain often associated with prolonged static postures, repetitive movements, and poor positioning. This has been the norm because there has not been adequate focus on equipment designed with the practitioner's musculoskeletal health in mind There are many chairs designed to enhance the comfort of the patient/subject; however, very few, if any, have been design to meet the health needs of the practitioner.

Therefore, a need exists to overcome the problems with the prior art as discussed above.

### SUMMARY OF THE INVENTION

The invention provides a dual-chair system that overcomes the hereinafore-mentioned disadvantages of the heretofore-known devices and methods of this general type.

With the foregoing and other objects in view, there is provided, in accordance with the invention, a dual-chair assembly for supporting a practitioner and a subject during a treatment, the assembly including: a base; a subject chair supported by the base; and a practitioner chair rotatably coupled to the base.

In accordance with a further feature of the present invention, the practitioner chair is rotatably coupled to the base by an extension arm defining a separation distance between the subject chair and the practitioner chair.

In accordance with yet another feature of the present invention, the extension arm is formed as a telescoping arm having a selectively adjustable length.

In accordance with another feature of the present invention, the practitioner chair is mechanically coupled to and disposed a selectively adjustable distance from the base.

In accordance with another feature of the present invention, the practitioner chair is operably configured to rotate approximately 180 degrees about the subject chair.

In accordance with a further feature of the present invention, the practitioner chair: is coupled to the base via a pivot; and is operably configured to rotate within a plane about a roll axis defined by the pivot.

In accordance with yet another feature of the present invention, the practitioner chair is operably configured to rotate approximately 180 degrees within a plane about a roll axis defined by a practitioner chair pivot.

In accordance with yet another feature of the present invention, the practitioner chair is operably configured to independently adjust each of an elevation, a pitch angle, and a yaw angle of the practitioner chair.

In accordance with another feature, an embodiment of the present invention includes a practitioner control communicatively coupled to practitioner chair actuators, the practitioner control: including a user input interface operably configured for receiving a practitioner input, and as a result of the practitioner input, causing the practitioner chair actuators to perform at least one of: rotating the practitioner chair about the subject chair; adjusting an elevation of the practitioner chair; rotating a roll angle of the practitioner chair; rotating a pitch angle of the practitioner chair; and rotating a yaw angle of the practitioner chair.

In accordance with a further feature of the present invention, the practitioner control is formed as a user foot control disposed proximate a foot area of the practitioner chair.

In accordance with another feature of the present invention, the base is integral with the subject chair.

In accordance with a further feature of the present invention, the dual-chair assembly is operably configured to: adjust of an elevation of the subject chair; and rotate the subject chair about an axis defined by the base.

In accordance with another feature, an embodiment of the present invention includes a user control communicatively coupled to subject chair actuators, the user control: including a user input interface operably configured for receiving a user input, and as a result of the user input, causing the subject chair actuators to perform at least one of: adjusting an elevation of the subject chair; and rotating the subject chair about an axis defined by the base.

In accordance with yet another feature, an embodiment of the present invention includes a magnetic field massager assembly including: a plurality of coils; and a power source electrically coupled to the plurality of coils for inducing a magnetic field through a seat area of at least one of the practitioner chair and the subject chair.

In accordance with a further feature of the present invention, the base is fixedly attached to a ground surface.

In accordance with another feature, an embodiment of the present invention includes a magnetic table coupled to the practitioner chair.

In accordance with the present invention, there is provided a physician-patient dual-chair assembly including: a physician chair rotatably coupled to a base by an extension arm, the base: fixedly attached to a ground surface, and disposed a separation distance from the physician chair; a patient chair coupled to and supported by the base; and a physician control communicatively coupled to physician chair actuators, the physician control: including a user input interface operably configured for receiving a physician input, and as a result of the physician input, causing the physician chair actuators to perform at least one of: rotating the physician chair about the patient chair; adjusting an elevation of the physician chair; rotating a roll angle of the physician chair; rotating a pitch angle of the physician chair; and rotating a yaw angle of the physician chair.

In accordance with another feature of the present invention, the physician chair is operably configured to rotate approximately 180 degrees about the patient chair.

In accordance with yet another feature of the present invention, the physician chair: is coupled to the base via the physician chair pivot; and is operably configured to rotate within a plane about a roll axis defined by the physician chair pivot.

In accordance with a further feature, an embodiment of the present invention includes a user control communicatively coupled to patient chair actuators, the user control including a user input interface operably configured for receiving a user input and causing the patient chair actuators to perform at least one of: adjust an elevation of the patient chair; and rotate the patient chair about an axis defined by the base.

Although the invention is illustrated and described herein as embodied in a dual-chair system, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the practitioner chair. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of an exemplary dual-chair assembly in accordance with the present invention;

FIG. 2 is a perspective view of the dual-chair assembly of FIG. 1, shown without the subject chair;

FIG. 3 is a perspective view of the dual-chair assembly on FIG. 1, illustrating a practitioner chair and a subject chair of the dual-chair assembly in a vertically elevated position, in accordance with the present invention;

FIG. 4 is a perspective view of the dual-chair assembly of FIG. 1, illustrating an extension arm in an extended position, in accordance with the present invention;

FIG. 5 is a perspective view of the dual-chair assembly of FIG. 1, illustrating the practitioner chair rotated from one side of the subject chair to another side of the patient chair, in accordance with the present invention;

FIG. 6 is a perspective view of the dual-chair assembly of FIG. 1, illustrating the practitioner chair with an adjusted roll angle, in accordance with the present invention;

FIG. 7 is a perspective view of the dual-chair assembly of FIG. 1, illustrating the practitioner chair with an adjusted pitch angle, in accordance with the present invention;

FIG. 8 is a perspective view of the dual-chair assembly of FIG. 1, illustrating the practitioner chair with an adjusted yaw angle, in accordance with the present invention;

FIG. 9 is a block diagram illustrating components of the dual-chair assembly of FIG. 1, in accordance with the present invention; and

FIG. 10 is an enlarged, fragmentary view of massager coils of the dual-chair assembly of FIG. 1, in accordance with the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient dual-chair assembly with a practitioner chair mechanically coupled to a base of a subject/patient chair. Embodiments of the invention provide that the practitioner chair is selectively adjustable in yaw, pitch, and roll angles. In addition, embodiments of the invention provide that the practitioner chair is selectively rotatable along a 360 degree orbital path around the subject chair. Additionally, the practitioner chair is adjustable in height relative to the ground and adjustable in distance from the subject chair. Such features allow the practitioner to continuously coordinate and adjust his/her positions respective to the patient throughout the treatment procedure for optimal practitioner comfort, good posture, and beneficial musculoskeletal health, resulting in a more dynamic, variable therapy environment.

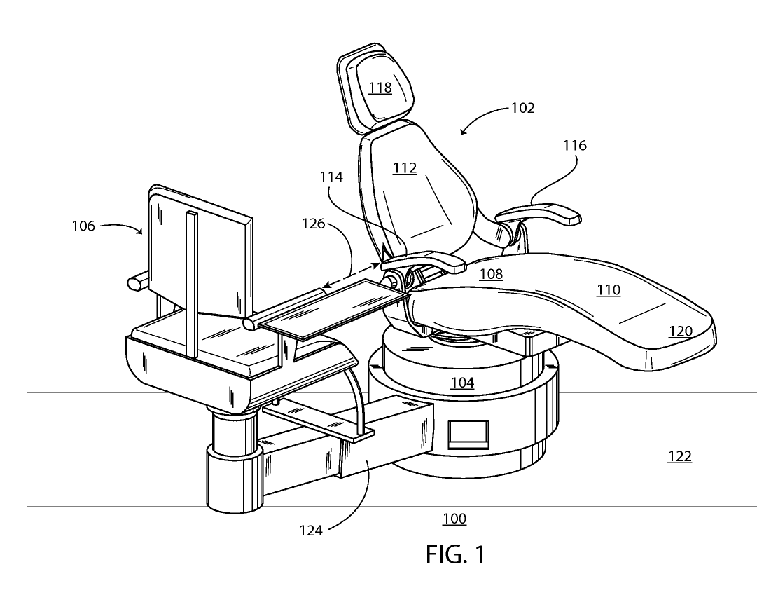

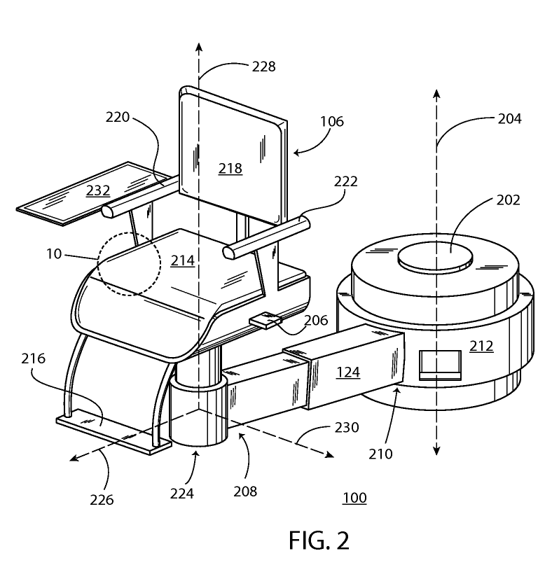

Referring now to FIGS. 1-2, one embodiment of the present invention is shown in perspective views. FIGS. 1-2 show several advantageous features of the present invention, but, as will be described below, the invention can be provided in several shapes, sizes, combinations of features and components, and varying numbers and functions of the components. The first example of a dual-chair assembly 100, as shown in FIGS. 1-2, includes a subject chair 102 supported by a base 104, and a practitioner chair 106 rotatably coupled to the base 104.

The subject chair 102 can be any chair operably configured to support a subject, such as a patient, a patron at a salon/barber shop, and the like. As used herein, the term “chair” is intended to indicate a support surface operably configured for at least one person to be supported thereon; including but not limited to support surfaces that may support the person in a seated position, a partially seated position, a standing position, or a generally horizontal or inclined resting position. For example, the term “chair” is intended to encompass horizontal support surfaces, such as surgical tables, massage tables, medical beds, and the like, as well as, support surfaces designed to support individuals in upright, seated positions. In the exemplary embodiment, the subject chair 102 includes a seat portion 108, a leg rest portion 110, a back rest portion 112, and a pair of opposing arm rests 114, 116. Other embodiments may not include each element of the exemplary subject chair 102; however, most subject chairs 102 will include at least a seat portion 108. Some embodiments of the subject chair 102 may also include a head rest 118 and a foot rest 120.

The base 104 is configured to support the subject chair 102, and, in some embodiments, provides one or more attachments points for coupling to the practitioner chair 106. In one embodiment, the base 104 extends upwardly from a ground surface 122. In another embodiment, the base 104 is fixedly attached to the ground surface 122 thereunder. As used herein, the term “fixedly attached” is intended to indicate a fastening to, an attachment to, or a placement on another structure or object so as to be firm and not readily movable. In one embodiment, the base 104 can be fixedly attached to the ground surface 122 with bolts, screws, and other fasteners. The base 104 is preferably fixedly attached to the ground surface 122 with a sufficient retention force so as to be able to support the subject chair 102, while also supporting the practitioner chair 106 coupled thereto. In another embodiment, the base 104 is not fastened to the ground surface 122, but includes a weight sufficient to support the practitioner chair 106 and the subject chair 102 coupled thereto. The base 104 can by any shape, size, and configuration. In one preferred embodiment, the base 104 is cylindrical-shaped, having a circular cross-section, so as to allow the practitioner chair 106 to be rotatingly coupled thereto and moveable along a circular path around a circumferential edge of the base 104. The base 104 can be made of any materials. In one embodiment, the base 104 is made of a rigid material, such as a metal or a rigid polymer-based material.

In one embodiment, the base 104 is integral with either of, or both of the subject chair 102 and the practitioner chair 106. As used herein, the term “integral” is defined as a direct, physical connection, or formed therewith as a unitary body. In another embodiment, the subject chair 102 is mechanically coupled to the base 104 through any mechanical coupling mechanism. Preferably, the subject chair 102 is rotatably coupled to the base 104 such that the subject chair 102 can be selectively rotated relative to a fixed position of the base 104. As is known in the art, the rotatable coupling can be accomplished by a subject chair pivot 202 interposed and connected between the subject chair 102 and the base 104. In one embodiment, the dual-chair assembly 100 is operably configured to rotate the subject chair 102 about an axis 204 defined by the base 104. In one embodiment, the axis 204 can be a pivot axis of the subject chair pivot 202. In another embodiment, the orientation and positioning of the subject chair 102 can be selectively adjusted with yaw, pitch, and roll rotations of the subject chair 102, similar to yaw, pitch, and roll rotations of an aircraft.

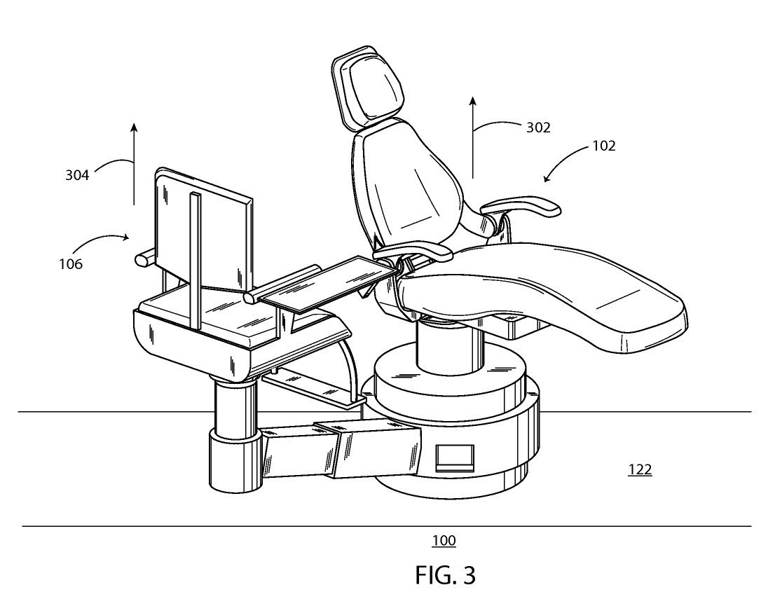

In yet another embodiment, the dual-chair assembly 100 is operably configured to adjust an elevation 302 of the subject chair 102 relative to the ground surface 122, as illustrated in FIG. 3. Actuators can be included within the subject chair 102 and/or within the base 104 to provide the motive force required to lift and rotate the subject chair 102. As is known in the art, the actuators can be any type of electric, mechanical, hydraulic, or pneumatic actuator, or any combination thereof. For example, in one embodiment, a hydraulic piston within the base 104 can allow the elevation 302 of the subject chair 102 to be adjusted.

Movement of the subject chair 102 can be controlled by a user control 206 that allows the practitioner to selectively adjust the orientation and positioning of the subject chair 102 for optimal reach during a treatment. In other embodiments, the user control 206 is disposed on or proximate to the subject chair 102, rather than the practitioner chair 106, to allow the subject, rather than the practitioner, to control orientation of the subject chair 102. In a preferred embodiment, the user control 206 is formed as a foot control in a foot area of the practitioner chair 106, such as a foot rest portion 216 of the practitioner chair 106. In another embodiment, the user control 206 is communicatively coupled to actuators of the subject chair 102 via, for example, a cable or a wire. The user control 206 can include a user input interface operably configured for receiving a user input to perform various functions, such as adjusting the elevation 302 of the subject chair 102 (see FIG. 3), and rotating the subject chair 102 about the axis 204. The user control 206 can include any user interface operable to receive a user input. For example, the user control 206 can include a keyboard, a keypad, a touchscreen, a button, a mouse, a joystick, a dial, or any other user input interface. Further, as a result of the user input, the user control 206 can send a signal to the actuators to cause the actuators of the subject chair 102 to perform at least one of the following: adjusting the elevation 302 of the subject chair 102, and rotating the subject chair 102 about the axis 204.

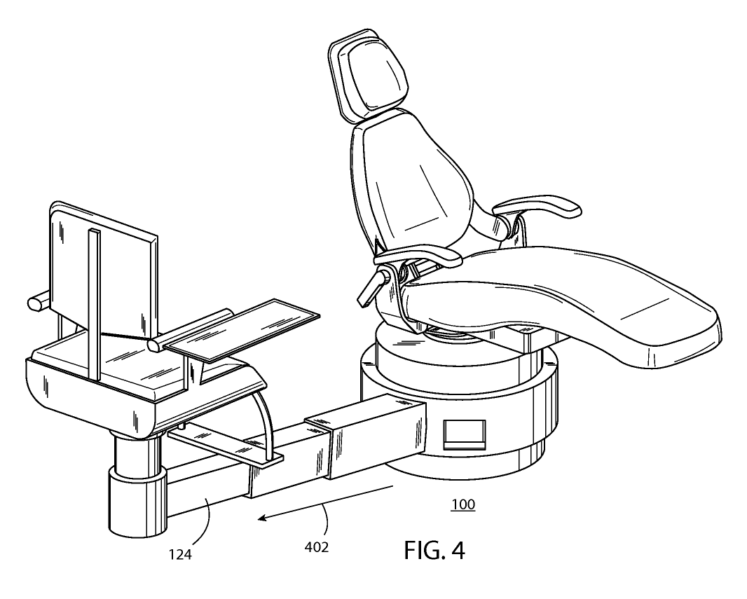

The practitioner chair 106 is rotatably coupled to the base 104 by an extension arm 124 defining a separation distance 126 between a peripheral edge of the subject chair 102 and a peripheral edge of the practitioner chair 106. In one embodiment, the extension arm 124 extends radially outward from a peripheral edge of the base 104. The practitioner chair 106 is coupled to an end 208 of the extension arm 124 that is distal from the base 104, while a proximal end 210 of the extension arm 124 is coupled to the base 104. The length of the extension arm 124 is preferably adjustable and more preferably adjustable by the practitioner via the foot control. The practitioner chair 106 can be said to be mechanically coupled to and disposed a selectively adjustable distance from the base 104. In one embodiment, the extension arm 124 is formed as a telescoping arm having a selectively adjustable length 402, as illustrated in FIG. 4. The extension arm 124 is preferably made of rigid support materials, such as a rigid polymer-based material or a metal material.

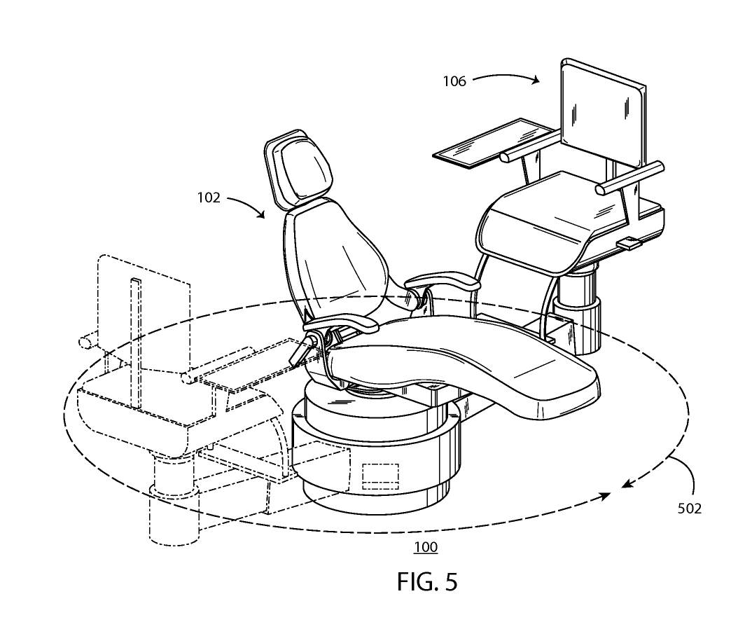

Still referring primarily to FIGS. 1-2, in one embodiment, the proximal end 210 of the extension arm 124 is coupled to a track 212. The track 212 can be circular-shaped and fixedly attached to or integral with the base 104. As used herein, the term “track” is intended to indicate a structure defining a continuous path coupled to the extension arm 124 so as to guide the extension arm 124 as it rotates about the subject chair 102. In one embodiment, the continuous path of the track 212 is concentric with the circular cross-section of the base 104. The track 212 provides a contact and support surface for the extension arm 124 to rotate the practitioner chair 106 about the subject chair 102. In one embodiment, the practitioner chair 106 is operably configured to rotate up to approximately 360 degrees within a generally horizontal plane about the subject chair 102, as illustrated in FIG. 5. Stated another way, the practitioner chair 106 follows a circular, orbital path 502 around the subject chair 102 and the base 104. Accordingly, the practitioner chair 106 allows the practitioner to, while remaining seated, selectively move along a continuous 360 degree circular path in either direction about the subject chair 102 for optimal positioning of the practitioner, relative to the subject, during treatment. In another embodiment, the practitioner chair 106 moves about the subject chair 102 along a continuous 180 degree arcuate path, rather than a full 360 degree circular path. Limiting movement to approximately 180 degrees may be considered a more efficient configuration because it allows the practitioner to move from one side of the patient to the opposing side of the patient, without the additional materials and design effort that may be required for a full 360 degree movement. In still further embodiments, the movement of the practitioner chair 106 about the subject chair 102 can lie outside of these ranges.

The practitioner chair 106 can be any chair operably configured to support a practitioner, such as a dentist, obstetrician, gynecologist, physician, or other medical professional, or a salon stylist, cosmetologist, barber, and the like. Accordingly, the practitioner chair 106 can be formed as a dentist chair, a physician chair, an OB/GYN chair, a cosmetologist chair, a barber chair, and the like. In one embodiment, the practitioner chair 106 includes a seat portion 214, the foot rest portion 216, a back rest portion 218, and a pair of opposing arm rests 220, 222. Other embodiments may not include each element of the exemplary practitioner chair 106; however, most practitioner chairs 106 will include at least a seat portion 214 and a back rest portion 218. In some embodiments, portions of the practitioner chair 106 and the subject chair 102 can include gel cushion elements for added comfort. For example, the arm rests 220, 222 may include gel cushion elements beneath the fabric covering.

In one embodiment, the practitioner chair 106 is coupled to the base 104 via a practitioner chair pivot 224 rotatably mounted to the distal end 208 of the extension arm 124. The practitioner chair pivot 224 allows the practitioner chair 106 to be rotated about one or more axes. As used herein, the term “pivot” is defined as an object mechanically coupled to a second object and on which the second object turns, oscillates, or rotates about. As is known in the art, the practitioner chair pivot 224 can be any type of pivoting mechanism, such as, for example, a hinge, a joint, a gimbal, and the like.

Still referring primarily to FIGS. 1-2, various rotational and elevational configurations of the practitioner chair 106 will be described with reference to FIGS. 3, 6, 7, and 8.

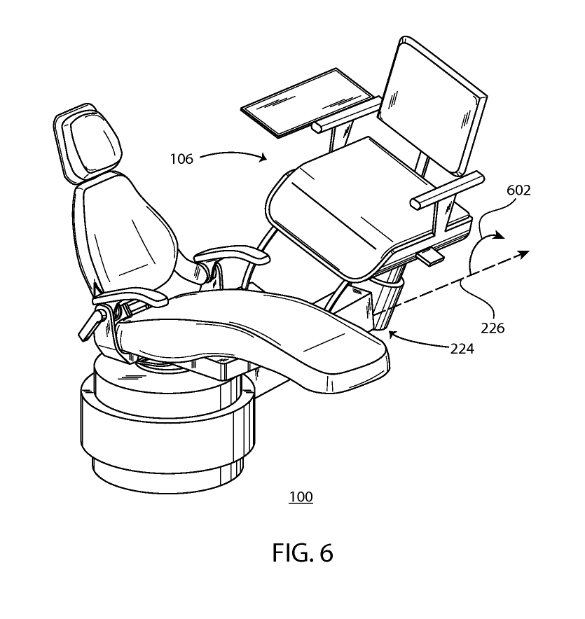

In one embodiment, the practitioner chair 106 is operably configured to rotate a roll angle 602 within a plane about a roll axis 226 defined by the practitioner chair pivot 224, as illustrated in FIG. 6. As used herein, the term “roll axis” is defined as an axis having its origin at a central pivot point of the practitioner chair pivot 224 and extending outwardly towards a front of the chair, e.g. towards the foot rest portion 216, as illustrated in FIGS. 2 and 6. The roll axis 226 is perpendicular to a yaw axis 228 and a pitch axis 230, similar to yaw, pitch, and roll axes of an aircraft. As used herein the term “roll angle,” is defined as an angle of rotation about the roll axis 226. In one embodiment, the practitioner chair 106 is operably configured to rotate approximately 180 degrees about the roll axis 226. In some embodiments, the practitioner chair 106 is operably configured to rotate approximately 90 degrees about the roll axis 226. For example, the practitioner chair 106 may be configured to rotate approximately 45 degrees in either direction from an absolute upright position of the practitioner chair 106. Other embodiments will be outside of these ranges.

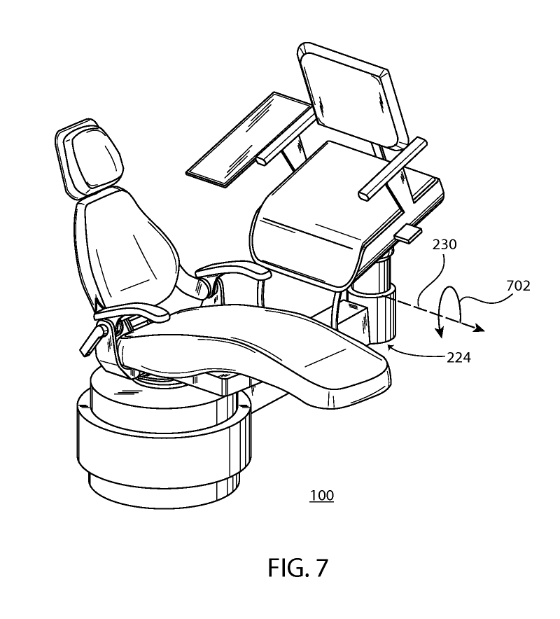

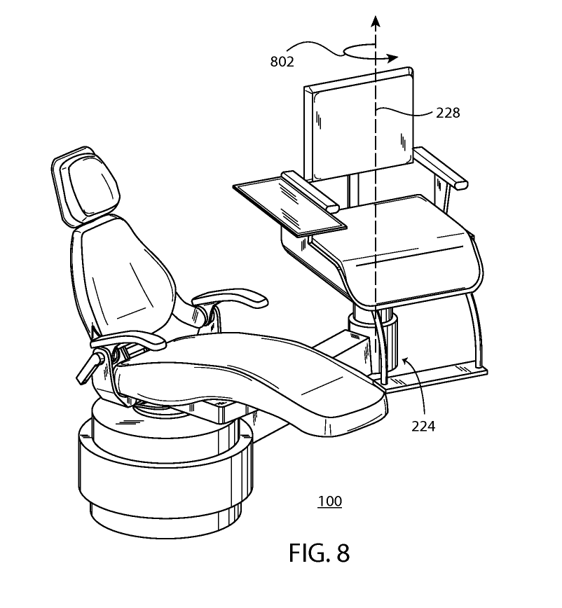

In another embodiment, the practitioner chair 106 is operably configured to independently adjust each of an elevation 304 (see FIG. 3), a pitch angle 702 (see FIG. 7), and a yaw angle 802 (see FIG. 8) of the practitioner chair 106. As used herein, the term “elevation” is defined as a height with respect to a support surface on which an object rests, such as the ground surface 122 on which the practitioner chair 106 rests. As used herein, the term “pitch angle” is defined as an angle of rotation about the pitch axis 230, where the pitch axis 230 is perpendicular to the yaw axis 228 and the roll axis 226. The “pitch axis” is defined as an axis having its origin at a central pivot point of the practitioner chair pivot 224 and extending laterally toward a side of the chair, e.g. toward the arm rest 222, as illustrated in FIGS. 2 and 7. As used herein, the term “yaw angle” is defined as an angle of rotation about the yaw axis 228, where the yaw axis 228 is perpendicular to the pitch axis 230 and the roll axis 226. The “yaw axis” is defined as an axis having its origin at a central pivot point of the practitioner chair pivot 224 and extending in a vertical direction relative to the chair, as illustrated in FIGS. 2 and 8. Allowing the practitioner to independently adjust elevation and various rotational angles provides the practitioner with a large range of freedom of movement to position himself in the most optimal manner for his musculoskeletal health and comfort during treatments, which can sometimes include many hours of repetitive movements and static postures.

Referring primarily to FIG. 2, in one embodiment, the practitioner chair 106 is coupled to a magnetic table 232 proximate to one side of an arm rest 220. The magnetic properties of the table 232 allow the table 232 to retain treatment tools, even when the practitioner chair 106 is titled or otherwise oriented in a position other than an upright position, which would otherwise cause the treatment tools to fall to the ground. The magnetic properties of the table 232 preferably include a magnetic force sufficient to retain the types of treatment tools typically used by the practitioner, yet not interfere with any other electrical or magnetic features of the assembly 100.

As with the subject chair 102, actuators can be included within the practitioner chair 106 to elevate and rotate the practitioner chair 106. As is known in the art, the actuators can be any type of electric, mechanical, hydraulic, or pneumatic actuator, or any combination thereof. For example, in one embodiment, a hydraulic piston within the practitioner chair 106 can allow the elevation 304 (see FIG. 3) of the practitioner chair 106 to be varied.

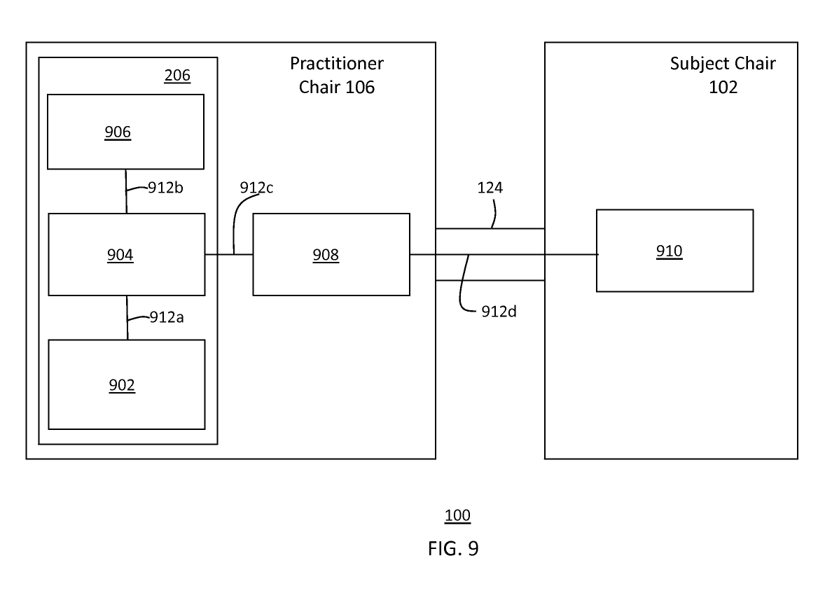

Referring now to FIG. 9, the dual-chair assembly 100 is presented in a block diagram illustrating an exemplary implementation. In one embodiment, the practitioner chair 106 includes a user input interface 902, a processor 904, memory 906, and practitioner chair actuators 908. The extension arm 124 is mechanically and, in some embodiments, electrically coupled to the practitioner chair 106 and the subject chair 102. The subject chair 102 includes subject chair actuators 910 that supply the motive force to move the subject chair 102.

The user input interface 902 functions to provide a user, such as the practitioner, a method of providing input to the dual-chair assembly 100. The user input interface 902 may also facilitate interaction between the practitioner and the dual-chair assembly 100. The user input interface 902 may be a keypad providing a variety of user input operations. For example, the keypad may include alphanumeric keys for allowing entry of alphanumeric information (e.g. telephone numbers, contact information, text, etc.). The user input interface 902 may include special function keys (e.g. volume control buttons, back buttons, home buttons, etc.), navigation and select keys, a pointing device, and the like. Keys, buttons, and/or keypads may be implemented as a touchscreen associated with a display. The touchscreen may also provide output or feedback to the user, such as haptic feedback.

Memory 906 associated with the assembly 100 may be, for example, one or more buffer, a flash memory, or non-volatile memory, such as random access memory (RAM). The assembly 100 may also include non-volatile storage. The non-volatile storage may represent any suitable storage medium, such as a hard disk drive or non-volatile memory, such as flash memory. In one embodiment, memory 906 may be memory located within the processor 904.

The processor 904 can be, for example, a central processing unit (CPU), a microcontroller, or a microprocessing device, including a “general purpose” microprocessing device or a special purpose microprocessing device. The processor 904 executes code stored in memory 906 in order to carry out operation/instructions input by the user via the user input interface 902, such as which direction to rotate, which axis to rotate about, and the degree of rotation desired. The processor 904 may provide the processing capability to execute an operating system, run various applications, and provide processing for one or more of the techniques described herein.

The actuators 908, 910 can be any type of electric, mechanical, hydraulic, or pneumatic actuator, or any combination thereof For example, the actuators 908, 910 can be formed as hydraulic motors, electric motors, hydro-electric actuators, compressed air engines, and the like. As used herein, the term “actuator” is defined as a device that supplies motive force to rotate, elevate, and otherwise move the practitioner chair 106 and/or the subject chair 102, or a portion thereof

In one embodiment, the components of the dual-chair assembly 100 are communicatively coupled via communication links 912 that can be wired or wireless. In another embodiment, the user input interface 902, the processor 904, and memory 906 can be considered the user control feature 206, allowing the user to control movement of the chairs 102, 106, as discussed above. In a preferred embodiment, the user control 206 is a practitioner control, allowing the practitioner to selectively adjust the movements of the practitioner chair 106 and/or the subject chair 102. In another embodiment, the user control 206 is communicatively coupled to the practitioner chair actuators 908 via the communication link 912, which can be, for example, a wire, a cable, or other electrically conductive medium. The user control 206 includes user input interface 902, which is operably configured for receiving a practitioner input, and, as a result of the practitioner input, the user control 206 causes the practitioner chair actuators 908 to perform at least one of the following: rotating the practitioner chair 106 about the subject chair 102; adjusting the elevation 304 of the practitioner chair 106; and rotating a roll angle 602, a pitch angle 702, and/or a yaw angle 802 of the practitioner chair 106.

In one embodiment, the user control 206 is formed as a user foot control disposed on or proximate to the foot rest portion 216 of the practitioner chair 106. This allows the practitioner to control movement of the chairs 102, 106 during treatment without use of his hands, eliminating the need to place any tools down, or interrupt treatment in order to user his hands to make any adjustments. The user foot control can be, for example, one or more foot pedals.

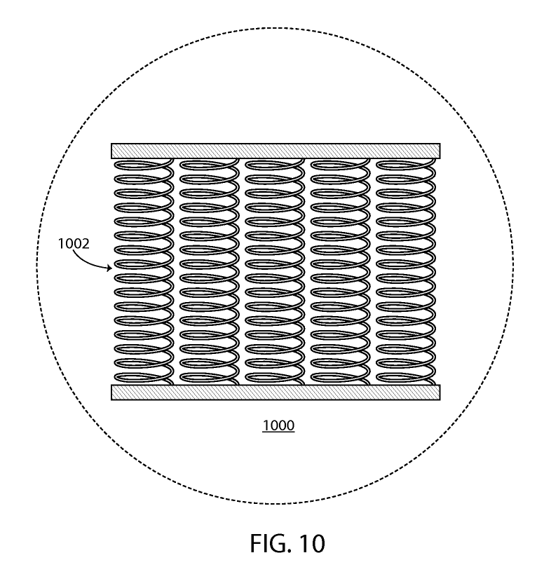

Referring now primarily to FIGS. 1-2 and 10, the dual-chair assembly 100 can include a massager assembly 1000, providing preventative stress relief, while enhancing circulation. In one embodiment, the massager assembly 1000 includes a plurality of conductive coils 1002 and a power source electrically coupled to the plurality of coils 1002 for selectively inducing a magnetic field through a seat area of the practitioner chair 106 and/or the subject chair 102. When the practitioner desires massage stimulation, he may enter an input via the user control 206 to induce the massaging magnetic field. In another embodiment, the massager assembly 1000 includes a logic controller that includes a 40,000 microFarad capacitor and a 2:1 step-up transformer capable of generating 250 volts. In other embodiments, a 10,000 microFarad capacitor can be used, without a step-up transformer. In yet another embodiment, the massager assembly 1000 includes multiple discrete discharge channels so that each of the plurality of coils 1002 may be activated individually. A standard 110 volt power source can provide the power to generate the appropriate electrical impulses. Any channel not being used for stimulation will be grounded to prevent electrical interference of the stimulated massager coil 1002. A frequency and amplitude of the electrical impulses will be determined by the logic controller. In one embodiment, the plurality of conductive coils 1002 can be formed as one or more arrays of overlapping copper insulated coils in series, with each array connected to a single channel associated with the logic controller. In a further embodiment, electrical current will flow through the logic controller with discharge of the capacitor and then out through a grounded lead in the logic controller. In one embodiment, activation of individual massager coils 1002 will occur in a rapid, controllable sequence that results in a magnetic field that can stimulate muscular tissue and, more particularly, stimulate motor neurons in the subcutaneous tissue perpendicular to the induced magnetic field.

A novel and efficient dual-chair assembly has been disclosed that includes a practitioner chair coupled to a subject chair. A user control, such as a foot control, allows various position and orientation adjustments to be made in both the subject and practitioner chair. Embodiments of the invention provide that the practitioner chair can be rotated up to 360 degrees around the periphery of the subject chair, which enables the practitioner to remain seated while orbiting the subject as needed during treatment. The subject chair lift/pivot allows independent rotation and elevation of the subject chair via the user control. A practitioner chair lift/pivot allows adjustments to be made with elevation and yaw, pitch, and roll rotations via the user control. Additionally, the user control allows the practitioner to alter the distance between the practitioner chair and the subject through extension and retraction of a selectively adjustable extension arm. The various movements of both chairs allow the practitioner to easily select the most optimal chair positions for ideal posture and positioning during treatment, which promotes musculoskeletal health of the practitioner and may allow practitioners to see as much as 5.5 more patients daily.

What is claimed is:

1. A dual-chair assembly for supporting a practitioner and a subject during a treatment, the assembly comprising:
a base;
a subject chair supported by the base; and
a practitioner chair rotatably coupled to the base.

2. The dual-chair assembly in accordance with claim 1, wherein:
the practitioner chair is rotatably coupled to the base by an extension arm defining a separation distance between the subject chair and the practitioner chair.

3. The dual-chair assembly in accordance with claim 2, wherein:
the extension aim is formed as a telescoping arm having a selectively adjustable length.

4. The dual-chair assembly in accordance with claim 1, wherein:
the practitioner chair is mechanically coupled to and disposed a selectively adjustable distance from the base.

5. The dual-chair assembly in accordance with claim 1, wherein:
the practitioner chair is operably configured to rotate approximately 180 degrees about the subject chair.

6. The dual-chair assembly in accordance with claim 1, wherein:
the practitioner chair:
is coupled to the base via a pivot; and
is operably configured to rotate within a plane about a roll axis defined by the pivot.

7. The dual-chair assembly in accordance with claim 1, wherein:
the practitioner chair is operably configured to rotate approximately 180 degrees within a plane about a roll axis defined by a practitioner chair pivot.

8. The dual-chair assembly in accordance with claim 1, wherein:
the practitioner chair is operably configured to independently adjust each of an elevation, a pitch angle, and a yaw angle of the practitioner chair.

9. The dual-chair assembly in accordance with claim 1, further comprising:
a practitioner control communicatively coupled to practitioner chair actuators, the practitioner control:
including a user input interface operably configured for receiving a practitioner input, and
as a result of the practitioner input, causing the practitioner chair actuators to perform at least one of:
rotating the practitioner chair about the subject chair;
adjusting an elevation of the practitioner chair;
rotating a roll angle of the practitioner chair;
rotating a pitch angle of the practitioner chair; and
rotating a yaw angle of the practitioner chair.

10. The dual-chair assembly in accordance with claim 9, wherein:
the practitioner control is formed as a user foot control disposed proximate a foot area of the practitioner chair.

11. The dual-chair assembly in accordance with claim 1, wherein:
the base is integral with the subject chair.

12. The dual-chair assembly in accordance with claim 1, wherein:
the dual-chair assembly is operably configured to:
adjust of an elevation of the subject chair; and
rotate the subject chair about an axis defined by the base.

13. The dual-chair assembly in accordance with claim 1, further comprising:
a user control communicatively coupled to subject chair actuators, the user control:
including a user input interface operably configured for receiving a user input, and
as a result of the user input, causing the subject chair actuators to perform at least one of:
adjusting an elevation of the subject chair; and
rotating the subject chair about an axis defined by the base.

14. The dual-chair assembly in accordance with claim 1, further comprising:
a magnetic field massager assembly including:
a plurality of coils; and
a power source electrically coupled to the plurality of coils for inducing a magnetic field through a seat area of at least one of the practitioner chair and the subject chair.

15. The dual-chair assembly in accordance with claim 1, wherein:
the base is fixedly attached to a ground surface.

16. The dual-chair assembly in accordance with claim 1, further comprising:
a magnetic table coupled to the practitioner chair.

17. A physician-patient dual-chair assembly comprising:
a physician chair rotatably coupled to a base by an extension arm, the base:
fixedly attached to a ground surface, and
disposed a separation distance from the physician chair;
a patient chair coupled to and supported by the base; and
a physician control communicatively coupled to physician chair actuators, the physician control:
including a user input interface operably configured for receiving a physician input, and
as a result of the physician input, causing the physician chair actuators to perform at least one of:
rotating the physician chair about the patient chair;
adjusting an elevation of the physician chair;
rotating a roll angle of the physician chair;
rotating a pitch angle of the physician chair; and
rotating a yaw angle of the physician chair.

18. The physician-patient dual-chair assembly in accordance with claim 17, wherein:
the physician chair is operably configured to rotate approximately 180 degrees about the patient chair.

19. The physician-patient dual-chair assembly in accordance with claim 17, wherein:
the physician chair:
is coupled to the base via the physician chair pivot; and
is operably configured to rotate within a plane about a roll axis defined by the physician chair pivot.

20. The physician-patient dual-chair assembly in accordance with claim 17, wherein:
a user control communicatively coupled to patient chair actuators, the user control including a user input interface operably configured for receiving a user input and causing the patient chair actuators to perform at least one of:
adjust an elevation of the patient chair; and
rotate the patient chair about an axis defined by the base.
